(19) United States
(12) Patent Application Publication
(10) Pub. No.: US 2014/0180126 A1
(43) Pub. Date: Jun. 26, 2014

(54) CATHETER WITH BALLOON AND IMAGING

(71) Applicant: VOLCANO CORPORATION, SAN DIEGO, CA (US)

(72) Inventors: Bret Millett, Folsom, CA (US); David Goodman, Carlsbad, CA (US)

(73) Assignee: VOLCANO CORPORATION, San Diego, CA (US)

(21) Appl. No.: 14/135,833

(22) Filed: Dec. 20, 2013

Related U.S. Application Data
(60) Provisional application No. 61/739,895, filed on Dec. 20, 2012.

(51) Int. Cl. A61B 1/00; A61F 2/958; A61M 25/10; A61B 8/12; A61B 8/00; A61B 17/12; A61B 5/00

(52) U.S. Cl. CPC A61B 1/00082; A61B 17/12109; A61B 17/12136; A61F 2/958; A61B 1/00165; A61B 5/0097; A61B 8/12; A61B 8/445; A61M 25/10; A61B 5/6853
USPC 600/467; 606/194; 600/478; 600/407; 600/476; 600/116

(57) ABSTRACT

The invention provides an intravascular catheter with an expandable member such as a balloon with an imaging device on a surface of the expandable member. The imaging device can use an optical fiber on a surface of the expandable member to image a treatment site. Since an image can be captured directly from the surface of the expandable member, an operator can examine the treatment site while positioning the expandable member. With the treatment site in view, the operator can deliver the treatment (e.g., inflate the balloon or deploy the stent) with confidence that it will be located properly.

### CROSS-REFERENCE TO RELATED APPLICATION

This application claims the benefit of, and priority to, U.S. Provisional Patent Application Ser. No. 61/739,895, filed Dec. 20, 2012, the contents of which are incorporated by reference.

### FIELD OF THE INVENTION

The invention generally relates to devices for cardiovascular intervention and particularly to balloon catheters with imaging devices.

### BACKGROUND

Some people suffer from an accumulation of fat, cholesterol, and other material on the walls of their blood vessels in deposits known as plaques. This condition, known as atherosclerosis or hardened arteries, often occurs with aging. Since the buildup of plaque in the blood vessels makes it difficult for blood to carry oxygen and nutrients to tissue throughout the body, plaque buildup can lead to tissue damage and death. Also, bits of plaque can break off and become lodged in the vessels. These plaque buildups can thus lead to heart attack and stroke.

Treatments for plaque buildups include balloon angioplasty and intravascular grafts. In balloon angioplasty, a catheter is used to carry a balloon to the site of plaque buildup. The balloon is inflated, forcing the expansion of the narrowed vessel. Intravascular grafts include tube-like stents and other expandable or coiled structures that are delivered to the affected vessel through the use of a balloon catheter. Stents help to hold the narrowed vessel open.

One significant challenge with balloons and stents is delivering them to the proper location within the vessel. A great amount of time and attention must be invested in establishing that the treatment device is precisely located at the affected site before it is deployed. Some approaches to this use an intravascular imaging technology, such as intravascular ultrasound (IVUS), to look for the target treatment site, and then move the treatment device into place. This can require, after positioning the treatment device, using ultrasound to look at the vessel outside of either end of the stent, and then adjusting the positioning as necessary. Not only is this iterative procedure time-consuming, it does not offer great precision. After a balloon is inflated or a stent is deployed, further imaging can reveal that the placement was poor, and that the device must be retracted and the procedure repeated. The time required this causes unnecessary patient discomfort and suffering as well as unnecessary risks of errors and infections.

### SUMMARY

The invention provides an intravascular catheter with an imaging device and a balloon. The imaging device can use an optical fiber on a surface of a balloon or other expandable member to image a treatment site, or the imaging device may be an IVUS transducer array located proximal to, distal to, or within an inflatable balloon on the catheter. Since an image can be captured directly from, or very close to, the location of the balloon, an operator can examine the treatment site while positioning the balloon. With the treatment site in view, the operator can deliver the treatment (e.g., inflate the balloon, deploy a stent, or both) with confidence that it will be located properly. In certain embodiments, a device of the invention has particular applicability in EVAR/TEVAR procedures (i.e., procedures for the repair of endovascular aortic aneurisms), where the balloon is used to shape a graft after the graft has been delivered; inflating the balloon within the graft can cause unwanted folds to disappear. A suitable imaging device can be provided by an optical fiber with a photoacoustic transducer and a fiber Bragg grating that use an optical signal to perform ultrasonic imaging, or by an IVUS transducer or array thereof. The optoacoustic imaging fiber can be disposed at a surface of the balloon. For example, the fiber may extend over a surface of an inflatable angioplasty balloon. Since imaging the site to be treated while deploying the treatment device allows an operator to confidently deploy the treatment to precisely the right location, atherosclerotic plaque can be treated without a time consuming and iterative positioning procedure and without any do-overs. Thus, a balloon with an imaging device on, within, or close to the balloon can minimize patient discomfort and suffering and avoid needless mistakes and complications.

In certain aspects, the invention provides a device for vascular intervention. The device includes a catheter with an expandable member such as a balloon and an image detector on a surface of the expandable member. In certain embodiments, the image detector is an optical-acoustic imaging fiber. An optical-acoustic imaging fiber can include an optical fiber and an acoustic-optic transducer. The fiber may extend over a surface of the catheter and balloon, or partially within the catheter shaft and partially over a surface of the balloon. In some embodiments, portion of the surface of the balloon can be adapted for use in image capture. For example, an ultrasonic signal can cause resonance of the balloon which can be used to modulate an electromagnetic signal, such as light being transmitted through an optical fiber.

The image detector may include one or more fiber Bragg grating. For example, a blazed fiber Bragg grating and an optical-to-acoustic transducer can be configured to use a source of optical energy to transmit acoustic energy into tissue. Such a structure may also or alternatively be used to receive an acoustic signal and modulate it onto an optical signal.

The device may further include a stent, for example, disposed around the balloon. The image detector may be provided as an optical fiber that extends substantially under the stent, between strands of the stent, or a combination thereof.

In related aspects, the invention provides a method for treating a patient by introducing an expandable member into a lumen within tissue, positioning the expandable member near a site to be treated, and imaging the site from a surface of the expandable member. The expandable member may be, for example, a balloon. The treatment site can be imaged directly while the balloon is inflating and may even be imaged while the balloon makes contact with the site. In certain embodiments, the imaging operation involves receiving ultrasonic energy from the tissue. The received image may be transmitted as an optical signal back down the catheter, e.g., from a distal portion to a proximal portion that extends outside of a patient's body. The optical signal may be an interferometric signal. Imaging the site may further include passing an electromagnetic signal through a fiber Bragg grating.

In some embodiments, imaging methods make use of one or more acoustic-to-optical transducers disposed at an expandable surface of the expandable member.

In another aspect, the invention provides a method that involves positioning a balloon to extend from a distal end of a treatment site to a proximal end of a treatment site within a lumen of a patient and viewing the treatment site from a location on a surface of the balloon between the distal and the proximal end of the treatment site during inflation of the balloon.

Aspects of the invention provide an imaging catheter with a balloon. The imaging can be provided by an IVUS transducer array. The IVUS imaging elements may be in, within, or near the balloon (e.g., just proximal to or just distal to the balloon). In certain embodiments, the balloon is a compliant balloon. An imaging device with a balloon can be used to deliver or to shape a stent or graft. Shaping a graft in situ may be particularly valuable in EVAR/TEVAR cases. Thus, device and methods of the invention aid in a properly repair aortic aneurisms.

An imaging catheter of the invention may include a catheter configured for insertion into vasculature of a patient, an intravascular ultrasound imaging device on a distal portion of the catheter, and a balloon on the distal portion of the catheter in fluid communication with an inflation lumen extending through the catheter. In some embodiments, the balloon comprises a compliant material that is flexible and elastic. The imaging device may be located to capture an IVUS image of a vessel through the compliant material. In certain embodiments, the imaging device captures an IVUS image at a frequency of less than 20 MHz, e.g., between 5 MHz and 15 MHz.

### BRIEF DESCRIPTION OF THE DRAWINGS

FIG. 1 depicts an apparatus with a catheter for vascular intervention.

FIG. 2 shows a cross-sectional view through a portion of a catheter.

FIG. 3. illustrates an acoustic-optical imaging fiber.

FIG. 4 gives a cross-sectional view of a catheter in some embodiments.

FIG. 5 gives a cross-sectional view of a catheter.

FIGS. 6A and 6B shows inflation of a balloon at a treatment site.

FIG. 7 shows a device for stent delivery with an imaging device on a surface of a balloon.

FIG. 8 shows a device being used to deploy a stent.

FIG. 9 shows a device for stent delivery with multiple imaging fibers.

FIG. 10 shows a cross sectional view through the dotted line in FIG. 9.

FIG. 11 shows use of an opto-acoustic imaging element.

FIG. 12 shows a catheter for intravascular ultrasound (IVUS) while using a balloon.

FIG. 13 shows advancement of a device towards a site for treatment.

FIG. 14 depicts imaging a treatment site with a device of the invention.

FIG. 15 depicts inflating a balloon 1 while imaging.

FIG. 16 illustrates use of a device of the invention to “iron out” a graft.

FIG. 17 shows a phased-array catheter with a balloon.

### DETAILED DESCRIPTION

Embodiments of the invention provide an intravascular catheter with an expandable member, such as a balloon, with an imaging device on a surface of the expandable member. Any suitable expandable member may be provided with an imaging device. In certain embodiments, the expandable member is a balloon. By using an imaging device on a surface of a balloon, placement of the catheter may be monitored as it is occurring.

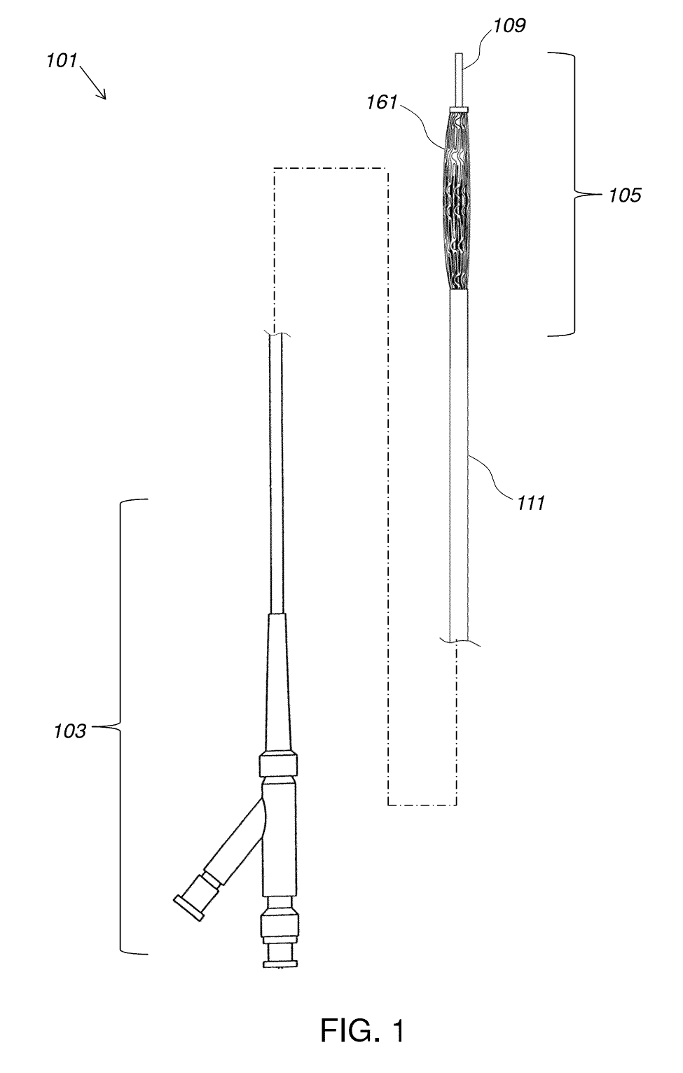

FIG. 1 shows a catheter 101 for cardiovascular intervention. Proximal portion 103 includes ports for guidewire and work tools. Catheter shaft 111 extends from proximal portion to distal portion 105. Distal portion 105 terminates in tip 109. Apparatus 101 is shown here having a stent 161 loaded thereon, although the invention provides intervention catheters that do not include a stent, such as catheters for balloon angioplasty. Devices for cardiovascular intervention are discussed in U.S. Pat. Nos. 6,830,559; 6,074,362; and U.S. Pat. No. 5,814,061, the contents of each of which are incorporated by reference.

A catheter 101 suitable for use with the methods of the invention will include an imaging element and a balloon. Catheters suitable for use with the invention typically include a guide wire lumen that allows the catheter to be directed to a point of treatment. The guide wire lumen may be a distinct guide wire lumen that runs the length of the catheter. In other embodiments, the guide wire lumen may only run a portion of the length of the catheter, e.g., a “rapid exchange” guide wire lumen. The guide wire lumen may be situated on top of the therapeutic delivery lumen or the guide wire channel could be side-by-side the therapeutic delivery lumen. In other cases, it may be possible to provide a fixed or integral coil tip or guide wire tip on the distal portion of the catheter or even dispense with the guide wire entirely. For convenience of illustration, guide wires will not be shown in all embodiments, but it should be appreciated that they can be incorporated into any of these embodiments.

Catheter bodies intended for intravascular introduction will typically have a length in the range from 50 cm to 200 cm and an outer diameter in the range from 1 French to 12 French (0.33 mm: 1 French), usually from 3 French to 9 French.

Catheter bodies will typically be composed of a biocompatible polymer that is fabricated by conventional extrusion techniques. Suitable polymers include polyvinylchloride, polyurethanes, polyesters, polytetrafluoroethylenes (PTFE), silicone rubbers, natural rubbers, and the like. Optionally, the catheter body may be reinforced with braid, helical wires, coils, axial filaments, or the like, in order to increase rotational strength, column strength, toughness, pushability, and the like. Suitable catheter bodies may be formed by extrusion, with one or more channels being provided when desired. The catheter diameter can be modified by heat expansion and shrinkage using conventional techniques. The resulting catheters will thus be suitable for introduction to the vascular system, often the coronary arteries, by conventional techniques.

Any suitable stent 161 may be used with device 101. One exemplary device for stent 161 is the Palmaz-Schatz stent, described, for example, in U.S. Pat. No. 4,733,665. Suitable stents are described in U.S. Pat. No. 7,491,226; U.S. Pat. No. 5,405,377; U.S. Pat. No. 5,397,355; and U.S. Pub. 2012/0136427, the contents of each of which are expressly incorporated herein by reference. Generally, stent 161 has a tubular body including a number of intersecting elongate struts. The struts may intersect one another along the tubular body. In a non-deployed state, the tubular body has a first diameter that allows for delivery of stent 161 into a lumen of a body passageway. When deployed, stent 161 has a second diameter and deployment of stent 161 causes it to exert a radially expansive force on the lumen wall. Methods of using stents are discussed in U.S. Pat. No. 6,074,362; U.S. Pat. No. 5,158,548; and U.S. Pat. No. 5,257,974, the contents of each of which are incorporated by reference. In some embodiments, stent 161 includes a shape-retaining or shape memory material such as nitinol and is self-expanding and thermally activatable within a vessel upon release. Such devices may automatically expand to a second, expanded diameter upon being released from a restraint. See, e.g., U.S. Pat. No. 5,224,953, the contents of which are incorporated herein by reference.

In certain embodiments, stent 161 is deployed through the use of expandable member disposed at distal portion 105 of catheter 111. An exemplary expandable member for the deployment of stent 161 is an inflatable balloon. Intravascular balloons and methods of use are known in the art. Such methods include the methods generally known as plain-old balloon angioplasty (POBA). Generally, an angioplasty balloon is deployed from a distal portion 105 of a catheter 111.

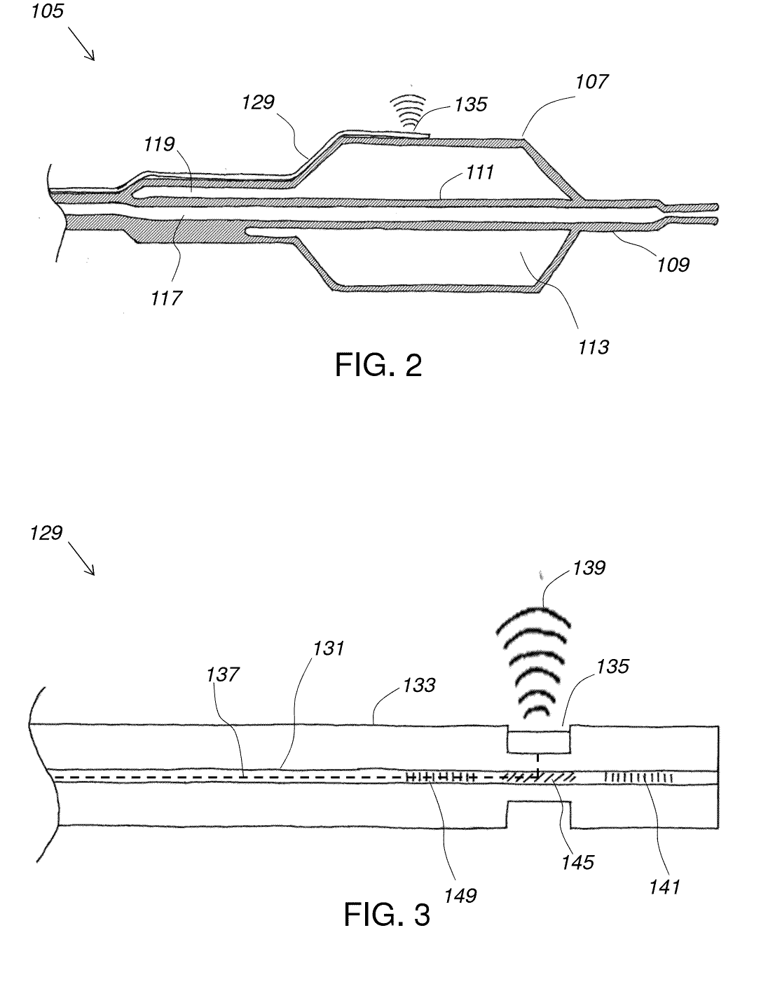

FIG. 2 shows a cross-sectional view through distal portion 105 of catheter 111. Guidewire lumen 117 extends through catheter shaft 111 to distal tip 109. Inflation channel 119 may generally be disposed along guidewire lumen 117 along a length of catheter 111. A plunging structure, or twist, may connect inflation lumen 113 to inflation channel 119 lying alongside guidewire lumen 117. Balloon 117 surrounds inflation lumen 113 which is in fluid communication with inflation channel 119. A surface of balloon 117 includes imaging device 135, shown here as a portion of imaging fiber 129.

Devices of the invention include a balloon on a catheter for delivery within a patient's vasculature. A balloon may be any suitable balloon known in the art such as, for example, a balloon used for POBA. A balloon may a compliant balloon or a non-compliant balloon. Balloon 107 is configured to be expandable, and may be used to deliver stent 161 or to open an obstructed vessel. Balloon 107 generally includes a strong flexible material and exhibits a narrow profile in an un-inflated state. Any suitable material may be used for balloon 107 including, for example, polyolefins such as polyethylene, polyvinyl chloride, polyesters such as polyethylene terephthalate (PET) and polybutylene terephthalate (PBT) and copolyesters, polyether-polyester block copolymers, polyamides, polyurethane, poly(ether-block-amide) and the like. Balloons are described in U.S. Pat. No. 7,004,963; U.S. Pub. 2012/0071823; and U.S. Pub. 2008/0124495, the contents of each of which are incorporated by reference. In some embodiments, the balloon will be constructed from a high-compliance material that is able to withstand pressures on the order of 6 to 10 atm. The expanding element may additionally include surface features such as ridges, studs, fins or protrusions to facilitate disruption of thrombus.

In some embodiments, imaging device 135 is provided by imaging fiber 129 extending from a proximal portion 103 of apparatus 101. At proximal portion 103, imaging fiber 129 may be operably coupled to a control unit (not pictured) via an optical coupler. Imaging device 135 may include any suitable imaging technology known in the art. In certain embodiments, device 101 uses optical-acoustic transduction to perform ultrasound imaging using imaging fiber 129 and imaging device 135.

FIG. 3 shows an imaging fiber 129 configured for optical-acoustic imaging. Along fiber 129, a cladding 133 surrounds fiber core 131. Light 137 is transmitted from the control unit down a length of fiber 129. Within fiber core, fiber Bragg grating 149 partially reflects light 137. Also, where included, terminal fiber Bragg grating 141 reflects light. Additionally, blazed fiber Bragg grating 145 reflects light in a direction substantially radial to an axis of fiber 129. The radial portion of the path of light 137 extends to photoacoustic transducer 135. When light 137 impinges on photoacoustic transducer 135, phonons are generated, leading to thermal strain of photoacoustic transducer 135. Thus, photoacoustic transducer 135 uses incoming light 137 as an energy source to generate a longitudinal pressure wave 139. When distal portion 105 of catheter 101 is in a patient's vessel, pressure wave 139 can be used for ultrasonic imaging of material in the vessel, plaque, the vessel wall, surrounding tissue, other material, or a combination thereof. Parts of wave 139 that bounce back constitute the return signal that will contribute to the ultrasonic image data.

In some embodiments, this return signal impinges on photoacoustic transducer 135. The energy of return signal causes a vibration or deformation of photoacoustic transducer 135. This results in a change in length of light path 137. In some embodiments, the primary change in length of light path 137 is in the radial portion extending between photoacoustic transducer 135 and fiber core 131, substantially perpendicular to an axis of fiber 129. However, deformations in geometry of cladding 133 may result in a change of length of light path 137 in, for example, the region between fiber Bragg grating 149 and blazed fiber Bragg grating 145. Depending on a desired embodiment, one may be favored over the other by cladding a portion of fiber 129 in a material with different rigidity or changing proportions of the depicted elements.

Additionally, transducer 135 may be positioned within an annular gap or a channel cutaway in fiber 129 and this portion of fiber 129 may define a diameter that is different than (e.g., smaller than) an overall diameter of fiber 129. The fiber diameter at transducer 135 may be dimensioned to vibrate at a resonant frequency congruent with a frequency of an ultrasonic signal. This can provide a benefit in terms of significant sensitivity for detector 135. For example, an incoming ultrasonic signal can induce vibration of fiber 129 due, in part, to major resonance modes associated with dimensions of the fiber at the annular gap or channel at detector 135. This resonant vibration can deform a material of fiber 129 causing a change in a length of a path of light 137 that reflects through blazed fiber Bragg grating 145, thus enhancing a quality of an image signal.

Light reflected by blazed fiber Bragg grating 145 from photoacoustic transducer 135 and into fiber core 131 combines with light that is reflected by either fiber Bragg grating 149 or 141 (either or both may be including in various embodiments). The light from photoacoustic transducer 135 will interfere with light reflected by either fiber Bragg grating 149 or 141 and the light 137 returning to the control unit will exhibit an interference pattern. This interference pattern encodes the ultrasonic image captured by imaging device 135. The light 137 can be received into photodiodes within a control unit and the interference pattern thus converted into an analog electric signal. This signal can then be digitized using known digital acquisition technologies and processed, stored, or displayed as an image of the target treatment site. An incoming optical acoustical signal impinging on diodes creates an analog electrical signal which can be digitized according to known methods. Methods of digitizing an imaging signal are discussed in Smith, 1997, THE SCIENTIST AND ENGINEER'S GUIDE TO DIGITAL SIGNAL PROCESSING, California Technical Publishing (San Diego, Calif.), 626 pages; U.S. Pat. No. 8,052,605; U.S. Pat. No. 6,152,878; U.S. Pat. No. 6,152,877; U.S. Pat. No. 6,095,976; U.S. Pub. 2012/0130247; and U.S. Pub. 2010/0234736, the contents of each of which are incorporated by reference for all purposes.

In related embodiments, imaging fiber 129 operates without a blazed fiber Bragg grating and detects a change in path length between fiber Bragg gratings 149 and 141 associated by a strain induced on fiber 129 by the impinging sonic return signal. In some embodiments, separate imaging fibers 129 are used to send and to receive an ultrasonic image. Methods of opto-acoustic imaging using fiber Bragg gratings for use with the invention are discussed in U.S. Pat. No. 8,059,923 and U.S. Pub. 2008/0119739, the contents of which are incorporated by reference in their entirety.

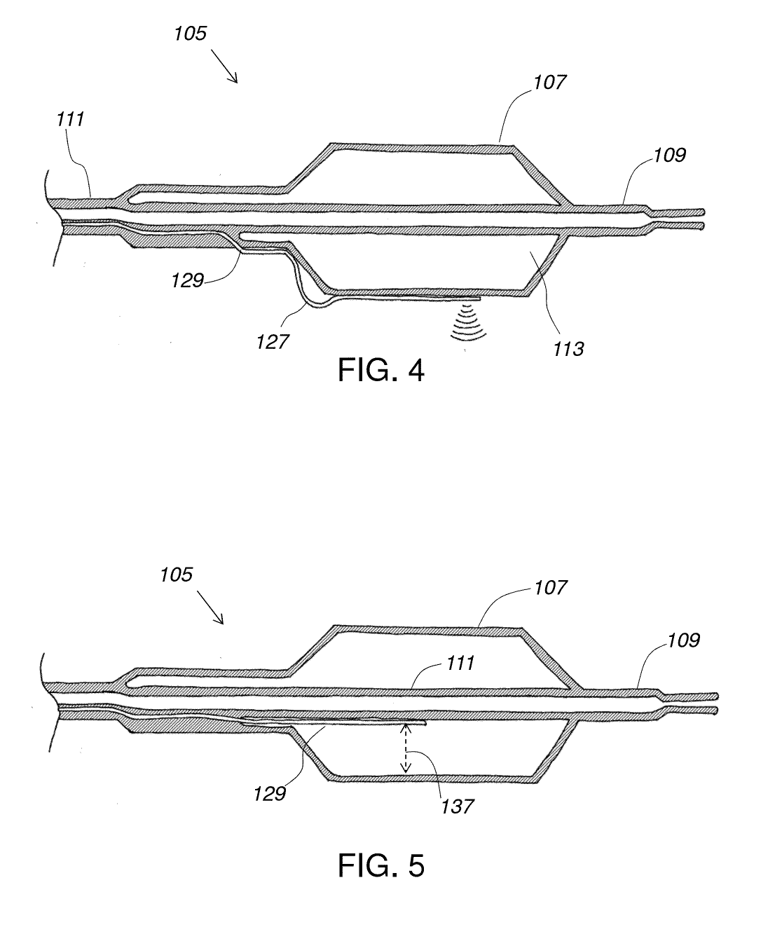

FIG. 4 gives a cross-sectional view of distal portion 105 of catheter 101 according to some embodiments. Imaging fiber 129 extends within an interior of catheter shaft 111 through a portion of its length and then traverses a wall of catheter shaft 111 and extends over a surface of balloon 107. This arrangement may provide particular benefit in protection of imaging fiber 129 along a substantial length of catheter shaft 111, while allowing a functional imaging detector 135 to be exposed to target site 151. Imaging fiber 129 may include an additional slack portion 127, for example, in the form of a hoop or a zigzag, so that when balloon 107 inflates, imaging fiber 129 does not get pulled off of the surface. Inflation lumen 113 carries an inflation fluid (e.g., air, gas, water, saline, a suspension, etc.) into balloon 107 to inflate it.

Balloon 107 may include any material that exhibits suitable strength and elasticity. Suitable materials may include polyvinyl chloride (PVC), cross-linked polyethylene (PET), nylon, or other polymers. In some embodiments, the balloon includes artificial muscle (electro-active polymer). Electro-active polymers exhibit an ability to change dimension in response to electric stimulation. The change may be driven by electric field E or by ions. Exemplary polymers that respond to electric fields include ferroelectric polymers (commonly known polyvinylidene fluoride and nylon 11, for example), dielectric EAPs, electro-restrictive polymers such as the electro-restrictive graft elastomers and electro-viscoelastic elastomers, and liquid crystal elastomer composite materials. Ion responsive polymers include ionic polymer gels, ionomeric polymer-metal composites, conductive polymers and carbon nanotube composites. Common polymer materials such as polyethylene, polystyrene, polypropylene, etc., can be made conductive by including conductive fillers to the polymer to create current-carrying paths. Many such polymers are thermoplastic, but thermosetting materials such as epoxies, may also be employed. Suitable conductive fillers include metals and carbon, e.g., in the form of sputter coatings. Electro-active polymers are discussed in U.S. Pat. No. 7,951,186; U.S. Pat. No. 7,777,399; and U.S. Pub. 2007/0247033, the contents of each of which are incorporated by reference.

Embodiments of the invention include an imaging device positioned on a surface of the balloon. In other embodiments, an imaging device includes material within the balloon. An ultrasonic signal causes motion of a surface of the balloon, and this motion is detected from the imaging device.

FIG. 5 gives a cross-sectional view of a catheter 101 in which imaging fiber 129 extends on a surface of catheter shaft 111 within the balloon. Using a blazed fiber Bragg grating as discussed above, light 137 is sent in a direction substantially perpendicular to an axis of catheter 111 to bounce off of an interior surface of balloon 107. Resonance of balloon 107 in response to an incoming ultrasonic signal causes a change in length of light path 137. An inside surface of balloon 107 may be mirrored or silvered or may have a mirror (e.g., a silvered plastic chip) affixed thereto. The changing length is detected by the interference between light reflected within fiber 129 and light reflected from the inside surface of balloon 107. Using a catheter 101 with an image detector on the surface of a balloon allows a physician to see a treatment site directly and to position the balloon accurately and precisely.

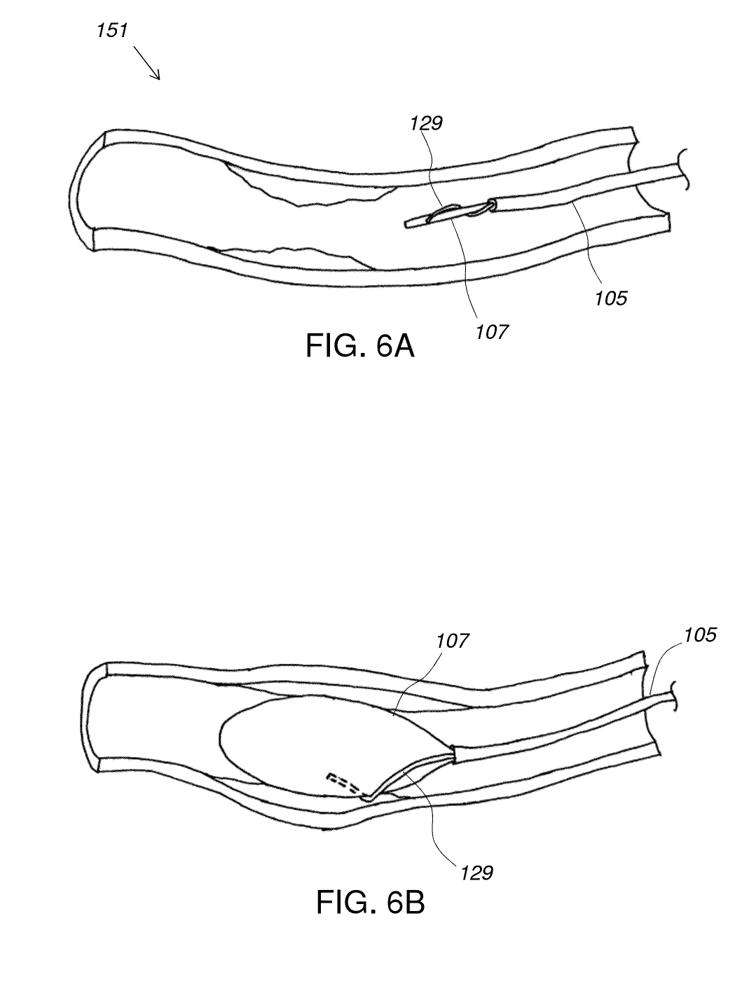

FIGS. 6A and 6B illustrates use of balloon 107 with imaging fiber 129 to view a treatment site 151. As distal portion 105 approaches a treatment site 151 (such as a region of a blood vessel affected by atherosclerotic plaque), a physician can view site 151 on a monitor of an associated medical imaging instrument (not pictured). Such vascular intervention procedures by catheter are often performed in specialized clinical environments known as cath labs. Cath labs and associated imaging instrumentation (e.g., IVUS and OCT instruments) are known in the art. For example, IVUS is discussed in U.S. Pat. No. 8,289,284; U.S. Pat. No. 7,773,792; U.S. Pub. 2012/0271170; U.S. Pub. 2012/0265077; U.S. Pub. 2012/0226153; and U.S. Pub. 2012/0220865. Optical-acoustic imaging structures (e.g., for imaging fiber 129) are discussed in U.S. Pat. No. 8,059,923; U.S. Pat. No. 7,660,492; U.S. Pat. No. 7,527,594; U.S. Pat. No. 6,261,246; U.S. Pat. No. 5,997,523; U.S. Pub. 2012/0271170 and U.S. Pub. 2008/0119739. The contents of each of these patents and publications are incorporated by reference in their entirety for all of their teachings and for all purposes.

As shown in FIG. 6A, distal portion 105 of catheter 111 is advanced through the vessel towards treatment site 151. Using, for example, IVUS or optical-acoustic imaging, the vessel wall is viewed to monitor for the location of atherosclerotic plaques. Monitoring a position of catheter 101 may also be optionally combined with use of standard x-ray angiographic techniques. When balloon 107 is positioned at the target treatment site, it is inflated, as shown in FIG. 6B, thus opening a passageway for blood to flow past the stenosized (narrowed) portion of the vessel. Balloon 107 may also be optionally used to deploy a stent.

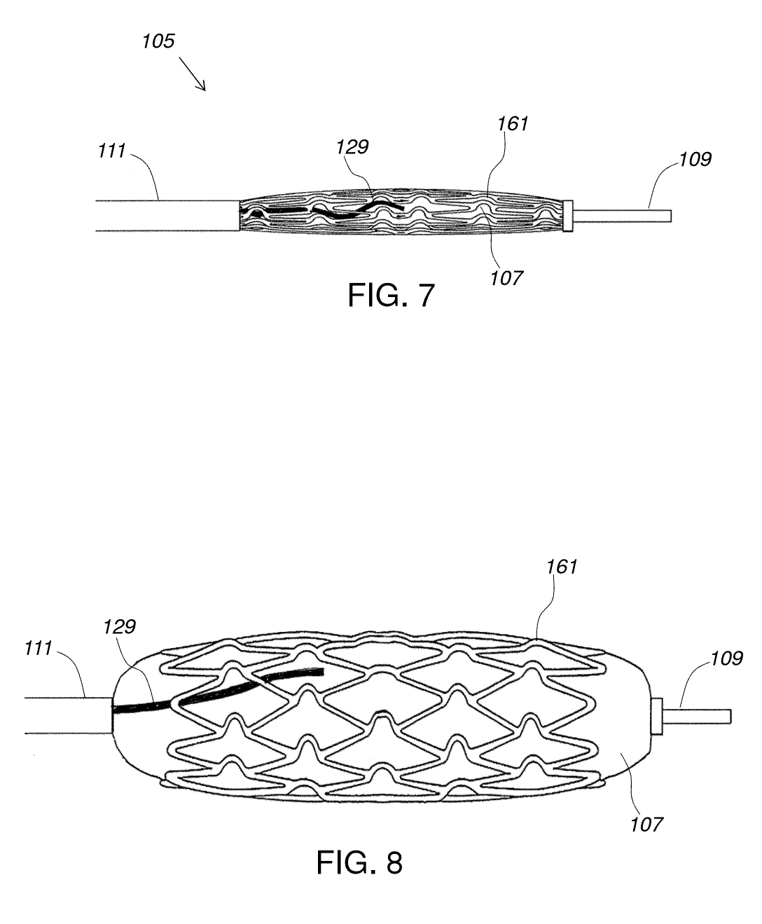

FIG. 7 shows use of catheter 101 to deliver a stent 161 into a vessel. As shown in FIG. 7, proximal portion of catheter shaft 111 has balloon 107 surrounded by stent 161 near distal tip 109. Imaging fiber 129 is disposed on a surface of balloon 107 (e.g., substantially as shown in FIG. 2).

FIG. 8 shows catheter 101 and balloon 107 as depicted in FIG. 7 in a deployed state. Balloon 107 has been inflated via inflation lumen 113. Stent 161 has been pushed into its expanded state. If inside of a vessel at treatment site 151, stent 161 will then remain in place when balloon 107 is deflated and catheter 111 is withdrawn and removed from the patient. FIG. 8 depicts a single imaging fiber 129 extending over a surface of balloon 107. This arrangement is provided and may be desired in some embodiments. However, any number of imaging fiber 129 may be included, and imaging device 135 of each of a plurality of imaging fiber 129 may be at the same, or different, distances away from distal tip 109. Balloon 107 may have any suitable number of imaging fiber 129 and any suitable number of imaging device 135, in part because any one imaging fiber 129 may include one or a plurality of imaging device 135. It may be preferred to have a number of imaging fiber 129 to visualize more completely treatment site 151. For example, balloon 107 may have 2, 3, 4, 5, 6, 7, 8, 9, 10, 12, 15, 20, 25, 30, 50, 64, 72, 80, 100, hundreds, etc., of imaging fiber 129.

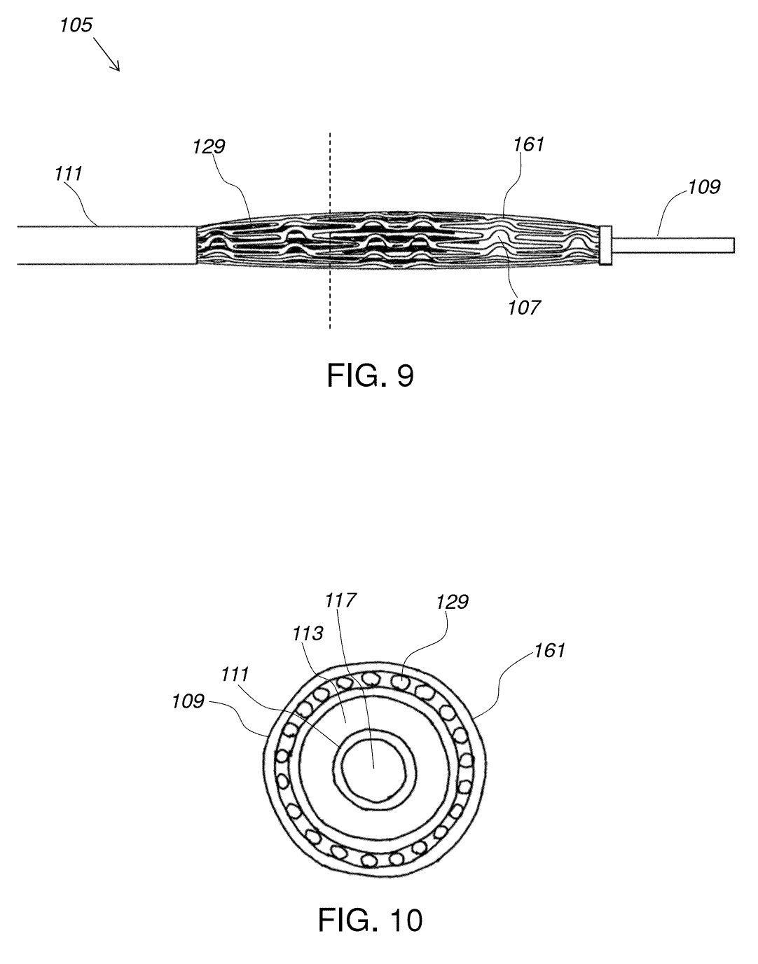

FIG. 9 shows a catheter 111 with balloon 107 having a plurality of imaging fiber 129 disposed around balloon 107, shown here under stent 161.

FIG. 10 shows a cross sectional view through the dotted line in FIG. 9. As shown in FIGS. 9 and 10, catheter shaft 111 includes a central work lumen 117 (e.g., for a guidewire or other tools or imaging devices). Around a body of catheter shaft 111 is balloon 107, spaced away by inflation lumen 113 (although in a deflated state, balloon 107 may have any geometry, such as an irregular shape, and may be substantially compressed against a body of catheter 111). Disposed around a surface of a balloon 107 are a plurality of imaging fibers 129. Each imaging fiber 129 presents an image detector 135 facing substantially away from an axis of catheter 111. As shown in FIG. 10, optional stent 161 may be disposed around an outside of balloon 107.

The invention includes methods of providing an array of imaging fibers 129 that can be disposed around balloon 107 as shown in FIG. 10 and further provides methods of creating a plurality of image detectors 135 that are all oriented in a desired direction. In some embodiments, a plurality of substantially featureless optical fibers are arrayed in a sheet substantially parallel to one another. The sheet of fibers may be positioned on a sheet of material that may optionally have an adhesive on the surface. Additionally or alternatively, a cementing material may be applied to the sheet-like array of fibers e.g., an epoxy or a thermo-setting polymer or a curable polymer. The fibers 129 may be arrayed in substantially straight lines (e.g., by combing prior to application of adhesive or cement) or may be in other conformations. For example, introducing a wavy or zigzag pattern into a portion of the fibers 129 may give them slack, or “play”, that allows image detectors to stay in place on a surface of balloon 107 when balloon 107 is inflated. Once the fibers are so arrayed and held in place, the fiber Bragg gratings may then be formed in all of them. The fiber Bragg gratings may be formed by an inscribing method using a UV laser and may be positioned through the use of interference or masking. Inscribing and use of fiber Bragg gratings are discussed in Kashyap, 1999, FIBER BRAGG GRATINGS, Academic Press (San Diego, Calif.) 458 pages; Othonos, 1999, FIBER BRAGG GRATINGS: FUNDAMENTALS AND APPLICATIONS IN TELECOMMUNICATIONS AND SENSING, Artech (Norwood, Mass.) 433 pages; U.S. Pat. No. 8,301,000; U.S. Pat. No. 7,952,719; U.S. Pat. No. 7,660,492; U.S. Pat. No. 7,171,078; U.S. Pat. No. 6,832,024; U.S. Pat. No. 6,701,044; U.S. Pub. 2012/0238869; and U.S. Pub. 2002/0069676, the contents of each of which are incorporated by reference. Detectors 135 can then be introduced by grinding a channel into the surface of all of the fibers. If done with the fibers un-cemented, the fibers can be rolled over and the grinding continued so that each fiber has an annular channel extending around the fiber. Fiber Bragg grating 149, 141, both, others, or a combination thereof can be formed, as well as any desired number of blazed fiber Bragg grating 145 in each fiber 129 (see FIG. 3). A channel or cutaway can be formed for image detector and may optionally be filled with a photoacoustic transducer material. Suitable photoacoustic materials can be provided by polydimethylsiloxane (PDMS) materials such as PDMS materials that include carbon black or toluene. Imaging fibers and methods of making them are discussed in U.S. Pat. No. 8,059,923, the contents of which are incorporated by reference for all purposes.

In related aspects and embodiments, the invention provides systems and methods for imaging from within a balloon.

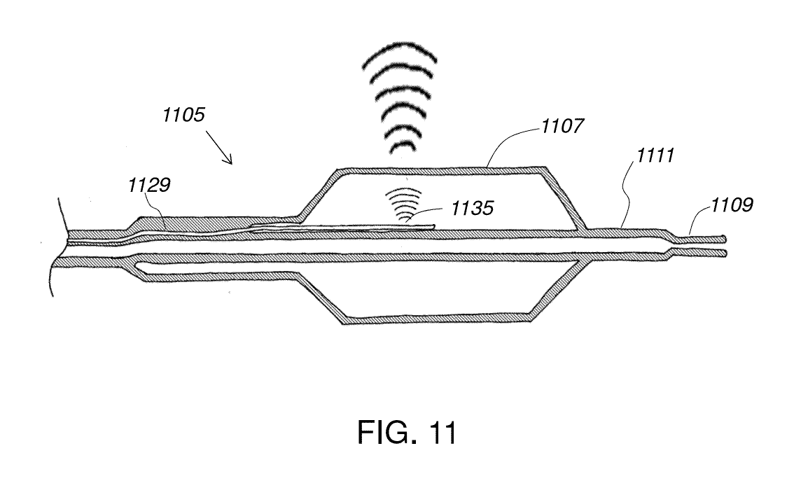

FIG. 11 shows use of an opto-acoustic imaging element 1135 for imaging through balloon 1107 on catheter 1105. Imaging element 1135 is preferably substantially as described above with respect to FIG. 3, as is imaging fiber 1120. Catheter 1105 has a main extended body 1111 terminating in distal tip 1109 with a lumen extending therethrough for a guidewire.

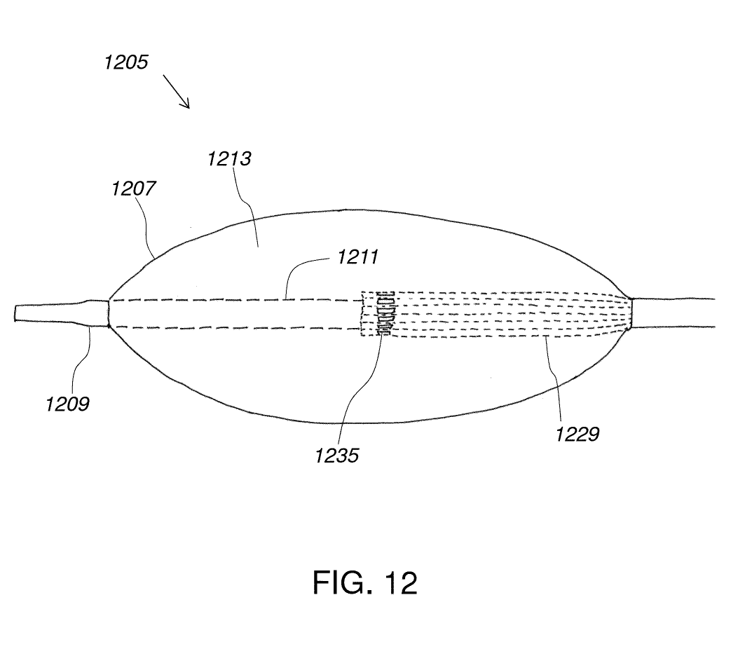

FIG. 12 illustrates an imaging catheter 1205 for imaging a vessel via intravascular ultrasound (IVUS) while using a balloon 1207. Imaging catheter 1205 includes an imaging assembly 1235. Preferably, imaging assembly 1235 includes any technology suitable for intravascular imaging such as technologies based on sound, light, or other media. Catheter 1205 may include an imaging portion 1229 (e.g., with conductor wires extending therethrough, surrounding an inflation lumen, guidewire lumen, or both, and extends to distal tip 1209.

As mentioned previously, in some embodiments, the imaging assembly 1235 is an IVUS imaging assembly. The imaging assembly can be a phased array IVUS imaging assembly, an pull-back type IVUS imaging assembly, or an IVUS imaging assembly that uses photoacoustic materials to produce diagnostic ultrasound and/or receive reflected ultrasound for diagnostics. IVUS imaging assemblies and processing of IVUS data are described for example in Yock, U.S. Pat. Nos. 4,794,931, 5,000,185, and 5,313,949; Sieben et al., U.S. Pat. Nos. 5,243,988, and 5,353,798; Crowley et al., U.S. Pat. No. 4,951,677; Pomeranz, U.S. Pat. No. 5,095,911, Griffith et al., U.S. Pat. No. 4,841,977, Maroney et al., U.S. Pat. No. 5,373,849, Born et al., U.S. Pat. No. 5,176,141, Lancee et al., U.S. Pat. No. 5,240,003, Lancee et al., U.S. Pat. No. 5,375,602, Gardineer et at., U.S. Pat. No. 5,373,845, Seward et al., Mayo Clinic Proceedings 71(7):629-635 (1996), Packer et al., Cardiostim Conference 833 (1994), “Ultrasound Cardioscopy,” Eur. J.C.P.E. 4(2):193 (June 1994), Eberle et al., U.S. Pat. No. 5,453,575, Eberle et al., U.S. Pat. No. 5,368,037, Eberle et at., U.S. Pat. No. 5,183,048, Eberle et al., U.S. Pat. No. 5,167,233, Eberle et at., U.S. Pat. No. 4,917,097, Eberle et at., U.S. Pat. No. 5,135,486, and other references well known in the art relating to intraluminal ultrasound devices and modalities. All of these references are incorporated by reference herein.

In other embodiments, the imaging may use optical coherence tomography (OCT). OCT is a medical imaging methodology using a miniaturized near infrared light-emitting probe, and is capable of acquiring micrometer-resolution, three-dimensional images from within optical scattering media (e.g., biological tissue). OCT systems and methods are generally described in Castella et al., U.S. Pat. No. 8,108,030, Milner et al., U.S. Patent Application Publication No. 2011/0152771, Condit et al., U.S. Patent Application Publication No. 2010/0220334, Castella et al., U.S. Patent Application Publication No. 2009/0043191, Milner et al., U.S. Patent Application Publication No. 2008/0291463, and Kemp, N., U.S. Patent Application Publication No. 2008/0180683, the content of each of which is incorporated by reference in its entirety.

FIG. 12 depicts a device 1205 for imaging and deploying a balloon 1207. Device 1205 includes a catheter 1211 with an extended body terminating at a distal tip 1209. Catheter 1211 may have one or more lumen therein such as, for example, a guidewire lumen to allow catheter 1211 to be guided to a treatment site. Preferably, catheter 1211 includes a separate inflation lumen allowing fluid 1213 (e.g., air) to be delivered to, and to inflate, balloon 1207. Catheter 1211 also includes an imaging device 1235. In FIG. 12, imaging device 1235 is depicted as being located within balloon 1207. It will be appreciated that imaging device 1235 may also be located just proximal to, or just distal to, balloon 1207. A portion 1229 of catheter 1211 carries requisite hardware for imaging assembly 1235 such as conductors or optical fibers. Imaging assembly 1235 may operate via any suitable imaging modality including, for example, ultrasound, opto-acoustic imaging, optical coherence tomography, or others.

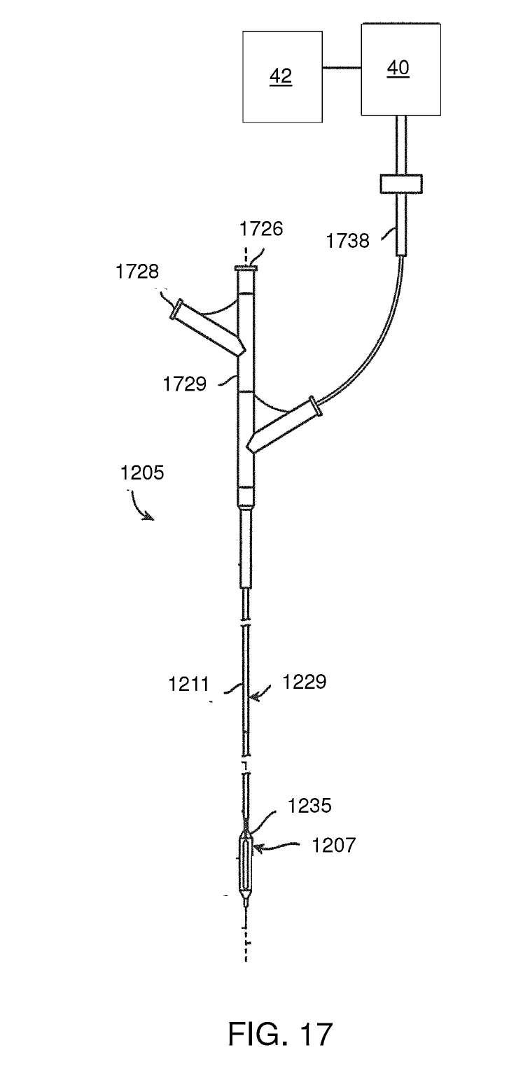

In some embodiments, e.g., as shown in FIGS. 12 and 17, the imaging assembly uses intravascular ultrasound (IVUS). IVUS-imaging catheters may be array-type catheters, i.e., as depicted in FIG. 12, or IVUS-imaging catheters may be pull-back type catheters. In some embodiments, an IVUS array is configured to image beyond the distal end of the catheter, i.e., “forward-looking” IVUS, or “FLIVUS.”

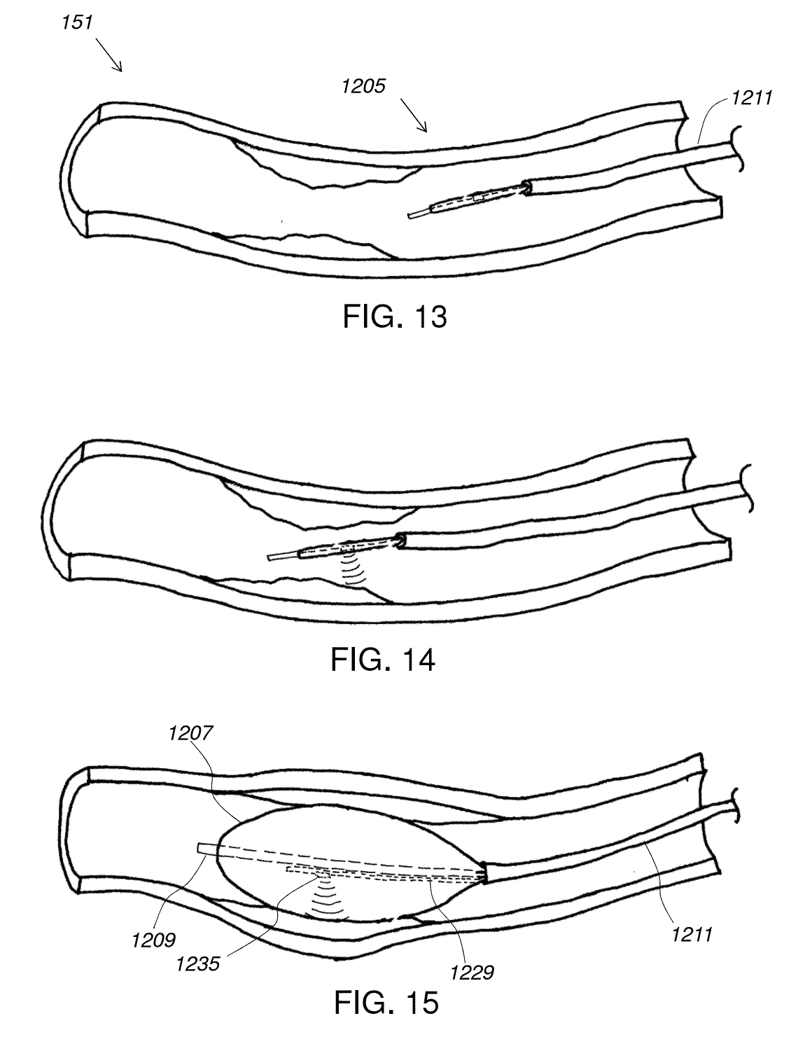

FIGS. 13-15 show use of device 1205 to treat a patient. In the depicted embodiments, device 1205 includes an IVUS catheter with a compliant balloon. In some embodiments, device 1205 is used in a EVAR/TEVAR procedure to help shape a stent or graft after it has been placed.

FIG. 13 shows advancement of a device 1205 towards a site 151 for treatment. While pictured here as a site of stenosis of a vessel, site 151 may be an aortic aneurism. Device 1205 or device 101 or any other device of the invention may have particular value in EVAR/TEVAR cases. In EVAR/TEVAR cases, a graft, such as a Y-shaped graft, is sometimes installed into the aorta (e.g., at the bifurcation). An imaging catheter with a compliant balloon as described herein may be particularly useful for “ironing out” the graft—pushing it from the inside to cause it to take a desired shape. Imaging can be accomplished by IVUS, photoacoustic imaging, or any other suitable modality on a catheter that also delivers a balloon, such as a compliant balloon.

FIG. 14 depicts imaging a treatment site 151 with a device of the invention.

FIG. 15 depicts inflating a balloon 1207 while imaging using imaging assembly 1235 at site 151. Imaging catheter 1229 generally describes the portions of catheter 1211 dedicated to imaging assembly 1235 (e.g., conductive cables). Terminus 1209 of catheter 1211 may be a shaped tip or open to provide access to a guidewire lumen.

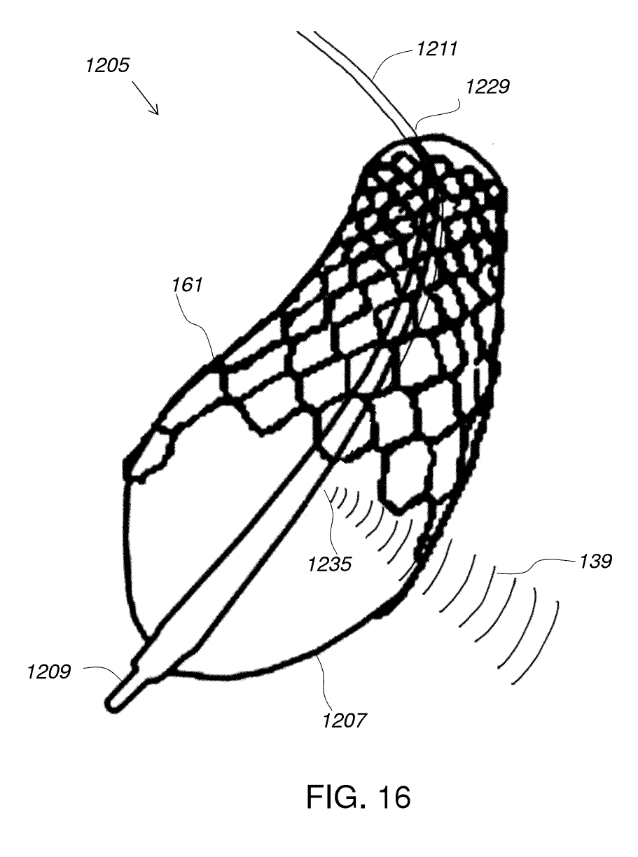

FIG. 16 illustrates use of a device of the invention to either deliver, or to “iron out”, a treatment device 161 such as a stent or a graft. Device 1205 is deployed within graft 161. Imaging assembly 1235 can image the treatment site by sound waves 139. Balloon 1207 is preferably a compliant balloon, although a non-compliant balloon may be preferred in some cases.

FIG. 17 shows a phased-array catheter 1205 that includes a distal portion 1209, a middle portion 1211, and a proximal portion 1729. The phase array catheter 1205 additionally includes a side arm 1728 for delivering fluid to expanding member 1207 as well as a guidewire lumen 1726 and connector 1738.

Imaging assembly 1235 is used to obtain ultrasound information from within a vessel. It will be appreciated that any suitable frequency and any suitable quantity of frequencies may be used. Exemplary frequencies range from about 5 MHz to about 80 MHz. In certain embodiments, the IVUS transducers operate at 10 MHz, or 20 MHz. In some embodiments, a frequency less than 20 MHz, such as between 5 MHz and 15 MHz, and preferably between 9 and 11 MHz, i.e., 10 MHz is used. Generally, lower frequency information (e.g., less than 40 MHz) facilitates a tissue versus blood classification scheme due to the strong frequency dependence of the backscatter coefficient of the blood. Higher frequency information (e.g., greater than 40 MHz) generally provides better resolution. Frequencies less than 20 MHz, such as between 5 MHz and 15 MHz, and preferably between 9 and 11 MHz, i.e., 10 MHz may be most useful for imaging large diameter vessels such as the aorta.

In some embodiments, the system additionally includes an image processing system 40 that receives image data from the imaging element 1235 and processes the image data to create new data that represents an image that can be displayed on display 42. The image processing system 40 can be constructed from a general use computer having a processor coupled to a non-transitory memory, however the image processor need not be a single stand-alone device, but may use distributed computing resources, such as cloud computing. While the system is depicted as a stand-alone collection of elements in FIG. 17, a system of the invention may also be constructed from elements that are not physically connected, using, e.g., wireless communication. Additionally, various controllers may communicate with the catheter via a network, e.g., via the internet.

Processing system 40 communicates with the imaging assembly 1235 by sending and receiving electrical signals to and from the imaging device 1235. Processing system 40 communicates with the via at least one electrical signal transmission member (e.g., wires or coaxial cable) within the device 1205. The processing system 40 can receive, analyze, and/or display information received from the imaging assembly 1235. It will be appreciated that any suitable functionality, controls, information processing and analysis, and display can be incorporated into the system 40. Further description of the interface module is provided, for example in Corl (U.S. patent application number 2010/0234736).

Catheter 1211 includes a imaging assembly 1235 preferably with a transducer housing. The transducer housing may be located at the distal end portion 1209 of device 1205 (e.g., within, adjacent to, or close to, balloon 1207). The imaging assembly 1235 can be of any suitable type, including but not limited to one or more advanced transducer technologies such as PMUT or CMUT.

The imaging assembly 1235 can include either a single transducer or an array. The transducer elements can be used to acquire different types of intravascular data, such as flow data, motion data and structural image data. For example, the different types of intravascular data are acquired based on different manners of operation of the transducer elements. For example, in a gray-scale imaging mode, the transducer elements transmit in a certain sequence one gray-scale IVUS image. Methods for constructing IVUS images are well-known in the art, and are described, for example in Hancock et al. (U.S. Pat. No. 8,187,191), Nair et al. (U.S. Pat. No. 7,074,188), and Vince et al. (U.S. Pat. No. 6,200,268), the content of each of which is incorporated by reference herein in its entirety. In flow imaging mode, the transducer elements are operated in a different way to collect the information on the motion or flow. This process enables one image (or frame) of flow data to be acquired. The particular methods and processes for acquiring different types of intravascular data, including operation of the transducer elements in the different modes (e.g., gray-scale imaging mode, flow imaging mode, etc.) consistent with the present invention are further described in U.S. patent application Ser. No. 14/037,683, the content of which is incorporated by reference herein in its entirety.

The acquisition of each flow frame of data is interlaced with an IVUS gray scale frame of data. Operating an IVUS catheter to acquire flow data and constructing images of that data is further described in O'Donnell et al. (U.S. Pat. No. 5,921,931), U.S. Provisional Patent Application No. 61/587,834, and U.S. Provisional Patent Application No. 61/646,080, the content of each of which is incorporated by reference herein its entirety. Commercially available fluid flow display software for operating an IVUS catheter in flow mode and displaying flow data is CHROMAFLO (IVUS fluid flow display software; Volcano Corporation).

While the imaging element 1235 is depicted as located proximal to the balloon 1207, the imaging element 1235 may also be located distal to, or within, balloon 1207. Co-located imaging and balloons are especially suitable for expanding member configurations, such as shown in FIG. 16, because the balloon material can be chosen such that it is essentially transparent to the ultrasound waves. Additionally, a catheter may be configured with a pull-back imaging element that is able to survey the entirety of the expanded member while it is expanded.

In certain embodiments, device 1205 has particular applicability in endovascular aneurysm repair (EVAR) procedure. Methods of the invention are useful with all EVAR related procedures, including without limitation, EVAR, hybrid EVAR, Common Iliac Artery EVAR, and Thoracic EVAR (TEVAR).

EVAR is typically conducted in a sterile environment, usually a theatre, under x-ray fluoroscopic guidance. The patient is usually administered an anesthetic prior to conducting the procedure. A puncture is then made with a needle in the femoral artery 202 of the groin. An introducer or vascular sheath is then inserted into the artery with a large needle, and after the needle is removed, the introducer provides access for guidewires, catheters, and other endovascular tools, such as the stent graft 161 used to treat the abdominal aneurysm. Once in place, the stent graft 161 acts as an artificial lumen for blood to flow through, and not into the surrounding aneurysm sac. This reduces the pressure in the aneurysm, which itself will usually thrombose and shrink in size over time.

Diagnostic angiography images or ‘runs’ are captured of the aorta to determine the location on the patient's renal arteries, so the stent graft can be deployed without blocking them. Blockage may result in renal failure, thus the precision and control of the graft stent deployment is extremely important. The main ‘body’ of the stent graft is placed first, follow by the ‘limbs’ which join on to the main body and sit on the Aortic Bifurcation for better support, and extend to the Iliac arteries. The stent graft (covered stent), once positioned, serves as an artificial lumen for blood to flow down, and not into the surrounding aneurysm sac. Accordingly, pressure is taken off the aneurysm wall, which itself will thrombose in time.

For certain occasions that the aneurysm extends down to the Common Iliac Arteries, a specially designed graft stent, named as Iliac Branch Device (IBD), can be used, instead of blocking the Internal Iliac Arteries, but to preserve them. The preservation of the Internal Iliac Arteries is important to prevent Buttock Claudication, and to preserve the full genital function.

### Incorporation by Reference

References and citations to other documents, such as patents, patent applications, patent publications, journals, books, papers, web contents, have been made throughout this disclosure. All such documents are hereby incorporated herein by reference in their entirety for all purposes.

### Equivalents

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A vascular intervention device comprising:
a catheter comprising an expandable member; and
a detector at a surface of the expandable member.

2. The device of claim 1, wherein the expandable member is a balloon.

3. The device of claim 2, wherein the detector comprises a portion of the surface of the balloon adapted to modulate an electromagnetic signal.

4. The device of claim 2 further comprising a stent disposed around the balloon.

5. The device of claim 4, wherein the detector comprises an optical fiber and a portion of the optical fiber is disposed between strands of the stent.

6. The device of claim 1, wherein the detector comprises a portion of an optical fiber and an acoustic-optic transducer.

7. The device of claim 1, wherein the detector comprises a portion of an optical fiber that includes one or more fiber Bragg gratings.

8. The device of claim 1, wherein the detector comprises a blazed fiber Bragg grating and an optical-to-acoustic transducer.

9. The device of claim 2, wherein the detector comprises a portion of a surface of the balloon adapted to resonate in response to a received acoustic signal and wherein the device further comprises an optical element to detect a resonance of the surface of the balloon.

10. A method for treating a patient comprising:
introducing an expandable member into a lumen within tissue;
positioning the expandable member near a site to be treated; and
imaging the site from a surface of the expandable member.

11. The method of claim 10, further comprising imaging the site while expanding the expandable member.

12. The method of claim 10, further comprising imaging the site while the expandable member makes contact with the site.

13. The method of claim 12, further comprising imaging the site at a point of contact from the surface of the expandable member while the expandable member makes contact with the site at the point of contact.

14. The method of claim 10, wherein imaging the site comprises receiving ultrasonic energy from the tissue.

15. The method of claim 10, wherein imaging the site further comprises passing an electromagnetic signal through a fiber Bragg grating.

16. A medical device comprising:
a catheter configured for insertion into vasculature of a patient;
an intravascular ultrasound imaging device on a distal portion of the catheter; and
a balloon on the distal portion of the catheter in fluid communication with an inflation lumen extending through the catheter.

17. The device of claim 16, wherein the balloon comprises a compliant material that is flexible and elastic.

18. The device of claim 17, wherein the imaging device is located to capture an IVUS image of a vessel through the compliant material.

19. The device of claim 18, wherein the imaging device captures an IVUS image at a frequency of less than 20 MHz.

20. The device of claim 19, wherein the frequency is between 5 MHz and 15 MHz.
